(19) United States
(12) Patent Application Publication
(10) Pub. No.: US 2010/0137433 A1
(43) Pub. Date: Jun. 3, 2010

(54) Production of Rosmarinic Acid from Spearmint and uses Thereof

(76) Inventors: Laima Kott, Heidelberg (CA); Ron Fletcher, Oakville (CA)

Correspondence Address: FULBRIGHT & JAWORSKI L.L.P., 600 CONGRESS AVE., SUITE 2400, AUSTIN, TX, 78701, US

(21) Appl. No.: 12/524,511

(22) PCT Filed: Jan. 23, 2008
(86) PCT No.: PCT/IB08/00978

§ 371 Date: Jan. 4, 2010

Related U.S. Application Data
(60) Provisional application No. 60/886,474, filed on Jan. 24, 2007.

(51) Int. Cl. A61K 31/235; A01H 5/00; A01H 1/02; A23F 3/00; C07C 69/76; C12P 7/62

(52) U.S. Cl. 514/532; 800/298; 800/260; 426/77; 426/597; 560/61; 435/135

(57) ABSTRACT

The present invention provides plants and plant tissues of spearmint (Mentha spicata) with enhanced rosmarinic acid levels, and extracts derived there from. Methods and compositions for production and use of rosmarinic acid from spearmint as a nutraceutical are also provided. In particular, an edible beverage derived from plant tissues of spearmint that comprise more than 77.5 mg/g rosmarinic acid on a dry weight basis, is provided, as well as methods for making such a beverage and for using it to treat an inflammatory or infectious disease.

### BACKGROUND OF THE INVENTION

The present application claims benefit of priority to U.S. Provisional Application Serial. No. 60/886,474, filed January 24, 2007, the entire contents of which are hereby incorporated by reference.

1. Field of the Invention

The present invention relates generally to the field of treatment of allergic reactions and diseases. More particularly, it concerns methods and compositions for production of food and nutraceutical substances comprising rosmarinic acid from spearmint plants, and their use in treating allergic reactions and diseases.

2. Description of Related Art

Rosmarinic acid (“RA”; α-O-caffeoyl-3,4-dihydroxyphenyl-lactic acid) is a polyphenolic antioxidant that has immunosuppressant, hepato and neuro-protective, anti-inflammatory, antibacterial and antiviral activities (Petersen and Simmonds, 2003; Iuvone et al., 2006; Renzulli et al., 2004; Yun et al., 2003; Tewtrakul et al., 2003). This compound is found primarily in plants of the mint family (Lamiaceae), including spearmint (Mentha spicata), oregano (Origanum vulgare), rosemary (Rosmarinus officinalis), and Red Perilla (Perilla frutescens). Recently, preclinical and clinical trials demonstrated that rosmarinic acid significantly reduces pulmonary inflammation, particularly as an effective modulator of symptoms associated with asthma and allergic reactions (Sanbongi et al., 2003; Sanbongi et al., 2004; Takano et al., 2004).

Bioavailability studies utilizing the oral administration of rosmarinic acid in different animal models has revealed that rosmarinic acid is absorbed, transported, modified, and is well tolerated. Topically applied rosmarinic acid is absorbed percutaneously and was transported throughout skin, blood, bone and muscle, while intravenously administered rosmarinic acid was found in the lung, spleen, heart and liver (Ritschel et al., 1989). Pharmacokinetic studies of rosmarinic acid in rats showed that the polyphenol is readily absorbed via the small intestine and reaches full concentration in the blood plasma within 30 minutes (Nakazawa and Ohsawa, 1998). The recovery of intact rosmarinic acid and metabolites in rat urine was 0.077% of the amount ingested (Nakazawa and Ohsawa, 1998). Rosmarinic acid was rapidly converted into mono-methylated and conjugated forms (glucuronide and sulfated). The conjugates were degraded into simpler phenolic forms (e.g. caffeic, coumaric, and ferulic acid conjugates and harmlessly excreted via the urine (Nakazawa and Ohsawa, 1998; Baba et al., 2004a). Metabolites attributed to rosmarinic acid were not found in bile (Baba et al., 2004a). Evidence also supports the rapid absorption of rosmarinic acid (Baba et al., 2004b) via the monocarboxylic acid transporter in the intestine (Konishi et al., 2005).

Several studies have demonstrated that rosmarinic acid is very effective in preventing oxidative damage to pulmonary tissue when induced by allergens. In a 2003 study, rosmarinic acid was administered to mice at a rate of 2 mg/day for 3 days prior to exposure to diesel dust particulates. Postmortem histological staining of lung tissue showed a marked decrease in protein and DNA damage compared to controls (Sanbongi et al., 2003). Rosmarinic acid was also found to inhibit proinflammatory factors interleukin 1β, keratinocyte monocyte chemoattractant proteins, macrophage inflammatory protein-2 (Sanbongi et al., 2003), the transport of neutrophils and eosinophils to the site of inflammation, and also reduced the accumulation of mucus cells in the airway supporting multiple modes of action.

RAD Natural Technologies (Petah Tikva, Israel) produces a product called Origanox™ WS from herbal extracts of oregano that contains about 7% rosmarinic acid. This product is sold as an anti-oxidant to the food industry, but a concentrated version containing up to 25% rosmarinic acid is available for use in cosmetic and therapeutic applications. Vitiva (Markovci, Slovenia) produces a product called AquaROX™ from dry rosemary (Rosmarinus officinalis) leaves, containing rosmarinic acids in various concentrations up to 70%. It is sold as an anti-microbial and anti-oxidant to the food industry. Meiji Seika Kaisha (Tokyo, Japan) has sponsored clinical trials on rosmarinic acid derived from red perilla (Perilla frutescens) (Hug, 2005).

Takano et al. (2004) and Osakabe et al. (2004) have described use of rosmarinic acid isolated from Red Perilla for relief of Hay Fever (seasonal allergic rhinoconjunctivitis) symptoms. Sanbongi et al, (2003, 2004) studied the effect of RA on lung injury and allergic inflammation. Inoue et al, (2005) reported on the effects of exposure to volatile components of rosemary on allergic asthma.

Wang et al. (2004) reported a RA content of 7.1-14.3 mg/g DW for Mentha spicata. Kosar et al. (2004) reported a RA content of 4.6 mg/g DW for Mentha spicata using a water extraction method. Fletcher et al. (2005a) reported a Mentha spicata clone (HMS-21) with enhanced RA levels of up to 60 mg/g DW. Fletcher et al. (2005b) also reported Mentha spicata clones with enhanced RA levels. McAuley (2002) reported additional Mentha spicata clones with enhanced RA levels of up to about 120 mg/g DW. However, as described below, these reported values of RA were based on an inaccurate spectrophotometric method, rather than HPLC. Thus, the highest RA concentration from the McAuley and Fletcher samples using the RA quantitation methods of the present invention was no more than 77.5 mg/g DW (see below).

U.S. Patent Publication 2006/0134236 describes anti-allergy compositions and related methods. U.S. Pat. No. 6,140,363 relates to use of rosmarinic acid and derivatives thereof as an immunosuppressant or an inhibitor of SH-2 mediated processes.

Alkam et al. (2007) reported that several doses of rosmarinic acid, following injection of Aβ25-35 into mice helped prevent the memory impairments (Y maze test and novel object recognition task) in untreated animals. Rosmarinic acid, at the effective lowest dose (0.25 mg/kg), prevented Aβ25-35-induced nitration of proteins, an indirect indicator of ONOO— damage, in the hippocampus, and also prevented nitration of proteins and impairment of recognition memory induced by ONOO-i.c.v.-injection. Co-injection of the non-memory-impairing dose of ONOO— with Aβ25-35 blocked the protective effects of RA (0.25 mg/kg). These results demonstrated that the memory protective effects of rosmarinic acid in the neurotoxicity of Aβ25-35 is due to its scavenging of ONOO—, and that daily consumption of rosmarinic acid may protect against memory impairments observed in Alzheimer's Disease.

Thus, a strong case has been made for the benefit of rosmarinic acid in a variety of clinical settings. The establishment of stable lines of Mentha spicata that produce high quantities of rosmarinic acid would be of benefit to anyone trying to isolate the compound and would greatly improve scientists' abilities to utilize rosmarinic acid for the benefit of consumers, the food processing industry, for use as a nutraceutical, and for patients suffering from an allergic condition or other conditions that may be alleviated by the presence of antioxidant activity. Spearmint plants and tissues comprising the levels of rosmarinic acid disclosed herein have not previously been reported.

### SUMMARY OF THE INVENTION

Thus, in one aspect, the present invention provides a tissue of a spearmint (Mentha spicata) plant, wherein the tissue comprises more than 77.5 mg/g rosmarinic acid to about 150 mg/g or more rosmarinic acid, on a dry weight basis. In particular embodiments, the tissue may comprise between more than 77.5 mg/g rosmarinic acid up to about 150 mg/g rosmarinic acid, for instance about 80-150 mg/g rosmarinic acid, or about 85 mg/g rosmarinic acid or more, 87 mg/g rosmarinic acid or more, 90 mg/g rosmarinic acid or more, or 92 mg/g rosmarinic acid or more, up to about 118 mg/g rosmarinic acid or more, or 150 mg/g rosmarinic acid, the rosmarinic acid content being calculated on a dry weight basis.

In certain embodiments, the tissue comprises a leaf, a stem, a flower, a seed, a cell, or a root, or parts or combinations thereof. In a particular embodiment, the invention includes a spearmint plant comprising tissue displaying such a rosmarinic acid content, or a part of such a plant. The tissue may further be defined as obtained from a plant containing genetic means for the expression of said more than 77.5 mg/g rosmarinic acid such as found in spearmint line 700B.

The spearmint plant part or tissue may further be defined as a leaf, stem, pollen, flower, seed, root, or cell. The tissue may be fully or partially dried and/or crushed or ground, and may be formulated in the following non-limiting embodiments, among others: as a teabag, as tea (i.e., as tea leaves or loose tea), as a caplet, as a tablet, or as another nutraceutical formulation comprising the tissue. In one embodiment, the invention includes a plant tissue culture comprising cells of such a spearmint plant, and material prepared from such a culture.

In another aspect, a tea or other beverage produced from the tea or teabag or other formulation, which beverage comprises at least about 90 mg or more of rosmarinic acid per 250 ml is included in the invention.

In yet another aspect, the invention relates to a method for producing rosmarinic acid, comprising cultivating a spearmint (Mentha spicata) plant, wherein the plant comprises tissue with more than 77.5 mg/g rosmarinic acid to about 150 mg/g or more rosmarinic acid, on a dry weight basis. In particular embodiments, the tissue may comprise between more than 77.5 mg/g rosmarinic acid up to about 150 mg/g rosmarinic acid, for instance about 80-150 mg/g rosmarinic acid, or about 85 mg/g rosmarinic acid or more, 87 mg/g rosmarinic acid or more, 90 mg/g rosmarinic acid or more, or 92 mg/g rosmarinic acid or more, up to about 118 mg/g rosmarinic acid or more, or 150 mg/g rosmarinic acid, the rosmarinic acid content being calculated on a dry weight basis. The method may also comprise isolating rosmarinic acid from the plant. In one embodiment, the invention also relates to a method for producing rosmarinic acid, comprising growing a tissue culture comprising cells of such a plant. The method may also comprise isolating rosmarinic acid from the plant tissue culture.

The invention also relates to a method of producing a beverage comprising rosmarinic acid, comprising contacting such plant tissue comprising between more than 77.5 mg/g rosmarinic acid up to about 150 mg/g rosmarinic acid, for instance about 80-150 mg/g rosmarinic acid, or about 85 mg/g rosmarinic acid or more, 87 mg/g rosmarinic acid or more, 90 mg/g rosmarinic acid or more, or 92 mg/g rosmarinic acid or more, up to about 118 mg/g rosmarinic acid or more, or 150 mg/g rosmarinic acid, with an edible liquid and allowing rosmarinic acid from the tissue to dissolve in the liquid. The liquid may comprise alcohol or water, or a combination thereof.

The invention also relates to a method for providing rosmarinic acid to a subject, comprising administering to the subject a spearmint tissue comprising between more than 77.5 mg/g rosmarinic acid up to about 150 mg/g rosmarinic acid, for instance about 80-150 mg/g rosmarinic acid, or about 85 mg/g rosmarinic acid or more, 87 mg/g rosmarinic acid or more, 90 mg/g rosmarinic acid or more, or 92 mg/g rosmarinic acid or more, up to about 118 mg/g rosmarinic acid or more, or 150 mg/g rosmarinic acid, or a composition comprising rosmarinic acid there from, such as a beverage comprising at least about 90 mg or more of rosmarinic acid per 250 ml. The method may further be defined as one wherein the subject is defined as comprising an inflammatory or infectious disease, and wherein the rosmarinic acid treats the inflammatory or infectious disease. Thus, the subject may suffer from nasal polyps, asthma, allergy, hay fever, seasonal allergic rhinitis, perennial allergic rhinitis, allergic rhinoconjunctivitis, eosinophilia, hypersensitivity, allergic conjunctivitis, eczema, food allergy, dermatitis, Alzheimer's Disease, among other diseases or conditions. The method may further be defined as one wherein the rosmarinic acid acts as an immunosuppressant, hepato- or neuro-protective, antibacterial, or antiviral.

In yet another aspect, the invention relates to a method for reducing the number of eosinophils at a site in a subject, comprising: administering to the subject the tissue comprising between more than 77.5 mg/g rosmarinic acid up to about 150 mg/g rosmarinic acid, for instance about 80-150 mg/g rosmarinic acid, or about 85 mg/g rosmarinic acid or more, 87 mg/g rosmarinic acid or more, 90 mg/g rosmarinic acid or more, or 92 mg/g rosmarinic acid or more, up to about 118 mg/g rosmarinic acid or more, or 150 mg/g rosmarinic acid, or a composition comprising rosmarinic acid there from, such as a beverage comprising at least about 90 mg or more of rosmarinic acid per 250 ml.

The invention further provides a method of producing seed, comprising crossing the spearmint plant wherein the plant comprises tissue with more than 77.5 mg/g rosmarinic acid to about 150 mg/g or more rosmarinic acid on a dry weight basis, for instance between more than 77.5 mg/g rosmarinic acid up to about 150 mg/g rosmarinic acid, for instance about 80-150 mg/g rosmarinic acid, or about 85 mg/g rosmarinic acid or more, 87 mg/g rosmarinic acid or more, 90 mg/g rosmarinic acid or more, or 92 mg/g rosmarinic acid or more, up to about 118 mg/g rosmarinic acid or more, or 150 mg/g rosmarinic acid, with itself or a second spearmint plant.

The invention also relates to a method of producing a spearmint plant comprising tissue with more than 77.5 mg/g rosmarinic acid to about 150 mg/g or more rosmarinic acid on a dry weight basis, comprising: preparing a progeny plant derived from a plant comprising tissue with more than 77.5 mg/g rosmarinic acid to about 150 mg/g or more rosmarinic acid on a dry weight basis by crossing the plant with a second spearmint plant, and obtaining a progeny plant comprising tissue with more than 77.5 mg/g rosmarinic acid to about 150 mg/g or more rosmarinic acid on a dry weight basis. In one embodiment, a plurality of progeny plants may be produced. Further, one or more of the obtained progeny plants may be selected based on rosmarinic acid content. In another embodiment, the progeny plant may be vegetatively propagated.

Also provided is a method for improving memory or impairing memory loss in a subject comprising administering to the subject the tissue of claim 1 or a composition comprising rosmarinic acid there from.

As used herein the specification, “a” or “an” may mean one or more. As used herein in the claim(s), when used in conjunction with the word “comprising,” the words “a” or “an” may mean one or more than one. As used herein, “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: HPLC tracing showing Rosmarinic acid obtained from tea (water extract).

FIG. 2: HPLC tracing comparing levels of rosmarinic acid obtained from teas prepared from a commercially available spearmint teabag and from spearmint line 700B. The HPLC traces are staggered on the same scale to illustrate the difference in rosmarinic acid concentrations between the 700B tea and the commercially available spearmint tea.

FIGS. 3A-3B: Allergen-induced broncho-constriction of subjects, hours post-challenge.

FIGS. 4A-4B: Allergen-induced airway inflammation of subjects-sputum eosinophil levels.

FIG. 5: Structure of rosmarinic acid.

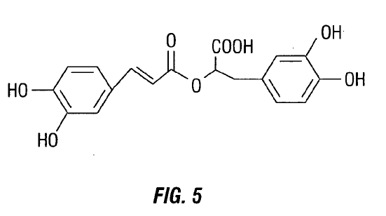

### DETAILED DESCRIPTION OF THE INVENTION

The current invention overcomes deficiencies in the prior art by providing plant tissue of Mentha spicata as a source of rosmarinic acid. This invention contemplates the use of such plant tissues displaying enhanced rosmarinic acid content, wherein the plant tissues comprise more than 77.5 mg/g rosmarinic acid, or about 80 mg/g or more, 85 mg/g or more, 87 mg/g or more, 90 mg/g or more, or 92 mg/g (about 9.2%) or more, up to about 118, or 150 mg/g rosmarinic acid on a dry weight basis, as a functional food or nutraceutical, for instance as a beverage such as a tea, capable of providing health benefits, and as a treatment for respiratory ailments, including, among others, Nasal polyps, Asthma, Allergy, Hay Fever, Seasonal Allergic Rhinitis, Perennial Allergic Rhinitis, Allergic Rhinoconjunctivitis, Eosinophilia, Hypersensitivity, Allergic Conjunctivitis, Eczema, Food Allergy, and Dermatitis. The method may further be defined as one wherein the rosmarinic acid acts as an immunosuppressant, hepato- or neuro-protective, antibacterial, or antiviral.

In certain embodiments, the invention comprises an edible or topically applied preparation of plant material, or an extract of such plant material, wherein the plant material comprises more than 77.5 mg/g, or about 80 mg/g or more, 85 mg/g or more, 87 mg/g or more, 90 mg/g or more, or 92 mg/g (about 9.2%) or more, up to about 118, or 150 mg/g rosmarinic acid on a dry weight basis. In particular embodiments, the plant material may be from spearmint (Mentha spicata) line 700B, comprising 87-118 or 87-150 mg/g rosmarinic acid on a dry weight basis.

The invention may comprise a plant part or tissue of spearmint, including a leaf, a stem, a flower, a seed, a cell, a tissue culture, or a root. The plant material may be dried and/or crushed or ground. In certain embodiments, the plant material comprises leaf tissue of spearmint, including dried and crushed leaf tissue. In a particular embodiment, the leaf or other tissue (which may be partially or largely dried and/or crushed or ground) is formulated as a loose tea or as tea leaves, or in a tea bag, for use in preparing an extract, such as a tea (i.e. beverage) or other water extract, of the spearmint leaves. In certain embodiments, the leaf or other tissue is dried and crushed or ground, and formulated as a caplet or tablet. A topical composition, comprising spearmint tissue comprising a rosmarinic acid content of at least about 8.7% or at least about 9% (DW) up to about 15%, is also a part of the invention.

The invention also relates to a nutraceutical, including a composition or an extract such as a tea, prepared from spearmint plant material. In particular embodiments, the extract, such as a tea, comprises at least about 90 mg rosmarinic acid per 250 ml, and may comprise, for instance, about 100 mg up to about 250 mg per 250 ml. In certain embodiments, the extract produced from spearmint, including a beverage such as a tea, may comprise 100 mg, 110 mg, 130 mg, 150 mg, or more rosmarinic acid per 250 ml. A teabag comprising such plant tissue is also an embodiment of the invention, as is tea (i.e. dried plant material; loose tea; tea leaves), and a caplet or tablet that comprises ground spearmint high in rosmarinic acid. In one particular embodiment, the invention comprises a teabag comprising about 2.5 g of dried and crushed or ground plant material, such as leaf tissue, and produces a tea comprising from about 90 mg to about 150 mg of rosmarinic acid per 250 ml serving. The composition may have antioxidant activity on its own, and also may be formulated with other components including one or more antioxidants, antimicrobials, nutrients, and/or flavorings.

The invention further relates to a method for producing a beverage comprising rosmarinic acid, comprising contacting plant tissue of spearmint (Mentha spicata) with an edible liquid such as water, and allowing rosmarinic acid from the tissue to dissolve in the liquid. To assist in the process of preparing an edible spearmint extract comprising rosmarinic acid, the temperature of the edible liquid may be varied. For instance, boiling water may be used. The composition may comprise water, as well as one or more other edible components, such as ethanol or another edible alcohol.

The invention also relates to a method for producing a pharmaceutical, nutraceutical, cosmetic, or other composition comprising rosmarinic acid, comprising growing and harvesting plant material from a spearmint plant, or a spearmint cell culture, and preparing an extract or other edible product from the plant material or plant part that comprises rosmarinic acid. The extract may be prepared, for instance, by using heated water, a mixture of an alcohol and water, such as a 50% ethanol/50% water (v/v) mixture, or by extraction with an alcohol such as ethanol. The extract may be used as a nutraceutical itself, or may comprise a portion of a further nutraceutical preparation or edible product.

The invention relates to use of a tea or other beverage or extract, of spearmint comprising about 8.7%-15% rosmarinic acid on a dry weight basis, including the use of an extract of spearmint for the manufacture of a medicament for the treatment of an inflammatory or infectious disease, such as nasal polyps, asthma, allergy, hay fever, Seasonal Allergic Rhinitis, Perennial Allergic Rhinitis, Eosinophilia, Hypersensitivity, Allergic Conjunctivitis, Eczema, Food Allergy, and Dermatitis, among other conditions. As a treatment, the medicament may also act as an immunosuppressant, a hepato- or neuro-protective, an antibacterial, or an antiviral.

The invention may also relate, in certain embodiments, to a method for the clinical treatment of allergic asthma, hay fever, allergic rhinitis, or other diseases or conditions, using plant material from Mentha spicata, for instance comprising an extract such as a tea or other extract or edible product, comprising at least about 100, about 125 mg, about 150 mg, or more, of RA derived from spearmint. In one aspect, the invention relates to a method to reduce the number of eosinophils at a site in a subject, wherein the eosinophils cause at least one allergic or inflammatory symptom such as, but not limited to, bronchoconstriction, eosinophilia, nasal congestion, sinus congestion, runny nose, cough, and itching. In certain embodiments, the extract comprises 90 mg or more, such as 100-150 mg, of RA. In a particular embodiment, the method may comprise drinking a tea made from Mentha spicata once, twice, or more times a day, such that at 300 mg or more of rosmarinic acid is ingested by a subject per day.

The invention further relates to a method for the clinical analysis of treatments for allergic asthma, hay fever, or allergic rhinitis, wherein the invention further demonstrates a superior clinical benefit from the use of Mentha spicata high in rosmarinic acid as a tea as compared to a second treatment.

The invention may further relate to mint plant breeding utilizing a spearmint line with enhanced rosmarinic acid content. Breeding techniques take advantage of a plant's method of pollination. There are two general methods of pollination: a plant self-pollinates if pollen from one flower is transferred to the same or another flower of the same plant. A plant cross-pollinates if pollen comes to it from a flower on a different plant. Mentha spicata is self-incompatible and is thus an out-crossing plant, generally requiring cross-pollination of differing genotypes, although it has been selfed with low efficiency.

Plants that have been pollinated and selected for type over many generations become homozygous at almost all gene loci and produce a uniform population of true breeding progeny, a homozygous plant. A cross between two such homozygous plants produces an agronomically uniform population of hybrid plants that are heterozygous for many gene loci. Conversely, a cross of two plants each heterozygous at a number of loci produces a population of hybrid plants that differ genetically and are not uniform. The resulting non-uniformity makes agronomic performance unpredictable.

### I. DEFINITION

Allele: Any of one or more alternative forms of a gene locus, all of which alleles relate to one trait or characteristic. In a diploid cell or organism, the two alleles of a given gene occupy corresponding loci on a pair of homologous chromosomes.

Backcrossing: A process in which a breeder repeatedly crosses hybrid progeny back to one of the parents, for example, a first generation hybrid (F₁) with one of the parental genotypes of the F ₁ hybrid.

Chromatography: A technique wherein a mixture of dissolved substances are bound to a solid support followed by passing a column of fluid across the solid support and varying the composition of the fluid. The components of the mixture are separated by selective elution.

Crossing: The pollination of a female flower of a plant, thereby resulting in the production of seed from the flower.

Cross-pollination: Fertilization by the union of two gametes from different plant genotypes.

Diploid: A cell or organism having two sets of chromosomes.

Emasculate: The removal of plant male sex organs or the inactivation of the organs with a chemical agent or a cytoplasmic or nuclear genetic factor conferring male sterility.

F₁ Hybrid: The first generation progeny of the cross of two plants.

Genetic Complement: An aggregate of nucleotide sequences, the expression of which sequences defines the phenotype in a plant, or components of plants including cells or tissue.

Genotype: The genetic constitution of a cell or organism.

Haploid: A cell or organism having one set of the two sets of chromosomes in a diploid.

Linkage: A phenomenon wherein alleles on the same chromosome tend to segregate together more often than expected by chance if their transmission was independent.

Marker: A readily detectable phenotype, preferably inherited in codominant fashion (both alleles at a locus in a diploid heterozygote are readily detectable), with no environmental variance component, i.e., heritability of 1.

Phenotype: The detectable characteristics of a cell or organism, which characteristics are the manifestation of gene expression.

Regeneration: The development of a plant from tissue culture.

Self-pollination: The transfer of pollen from the anther to the stigma of the same plant.

Tea: Dried plant material including, for instance, leaves, stems, and/or flowers (as loose tea; tea leaves), harvested and used in preparing an extract of the material such as a beverage; also including the extract such as a beverage prepared using such plant material.

Tissue Culture: A composition comprising isolated cells of the same or a different type or a collection of such cells organized into parts of a plant.

Vegetative propagation: Production of new plants (i.e., clones) from existing vegetative structures, such as, among others, rooted cuttings.

Genetic markers associated with enhanced rosmarinic acid content in spearmint may be identified. The presence and/or absence of a particular genetic marker allele in the genome of a plant exhibiting a favorable phenotypic trait may be made by any method using markers, examples of which, for examples, are Restriction Fragment Length Polymorphisms (RFLP), Amplified Fragment Length Polymorphisms (AFLP), Simple Sequence Repeats (SSR), Single Nucleotide Polymorphisms (SNP), Insertion/Deletion Polymorphisms (Indels), Variable Number Tandem Repeats (VNTR), and Random Amplified Polymorphic DNA (RAPD), among others known to those skilled in the art. If the nucleic acids from a plant are positive for a desired genetic marker, such as one linked to altered phenylpropanoid metabolism including enhanced RA content in Mentha spicata, the plant can possibly be selfed (although at low efficiency for this out-crossing plant), or it can be crossed with a plant with the same marker or with other desired characteristics to create a sexually crossed hybrid generation. Methods of marker-assisted selection (MAS) using a variety of genetic markers are known. The presence of the genetic marker may be correlated with a physiological or visible phenotype, such as germination in the presence of L-α-bromophenylalanine.

The present invention also provides, in another aspect, a genetic complement of the Mentha spicata variety designated line 700B that contains means for production of at least about 90 mg/g to about 150 mg/g of rosmarinic acid in plant tissue, on a dry weight basis. Means for determining such a genetic complement are well-known in the art. As used herein, the phrase “genetic complement” means an aggregate of nucleotide sequences, the expression of which defines the phenotype of a plant or a cell or tissue of that plant. By way of example, a plant is genotyped to determine a representative sample of the inherited markers it possesses. Markers are alleles at a single locus. They are preferably inherited in codominant fashion so that the presence of alleles at a locus is readily detectable, and they are free of environmental variation, i.e., their heritability is 1. This genotyping is preferably performed on at least one generation of the descendant plant for which the numerical value of the quantitative trait or traits of interest are also determined. The array of single locus genotypes is expressed as a profile of marker alleles at each locus. The marker allelic composition of each locus can be either homozygous or heterozygous. Homozygosity is a condition where both alleles at a locus are characterized by the same nucleotide sequence or size of a repeated sequence. Heterozygosity refers to different conditions of the gene at a locus. Preferred types of genetic marker for use with the invention are, for example, simple sequence repeats (SSRs), restriction fragment length polymorphisms (RFLPs), amplified fragment length polymorphisms (AFLPs), single nucleotide polymorphisms (SNPs), and isozymes (e.g. Shasany et al., 2005).

In one aspect, the invention relates to a method for identifying a spearmint plant or plant part such as a cell comprising enhanced levels of rosmarinic acid. Such a method may be performed by screening spearmint seeds germinated and grown in a solution of, for instance, the phenylalanine analogue, L-α-bromophenylalanine (e.g. about 0.7 mM), a putative inhibitor of phenylalanine ammonia lyase, the first enzyme in the phenylpropanoid pathway. Seeds may also be germinated and plants grown in the presence of rosmarinic acid itself (e.g. about 0.4-1 mM) to identify plants that display enhanced levels of rosmarinic acid. A tissue culture derived from such a plant is also an aspect of the invention.

Thus, spearmint seeds (e.g about 5 g, or about 50,000 seeds) may be surface sterilized and then washed with sterile water. The seeds may then be placed on pre-wetted sterile filter paper soaked in a solution of L-α-bromophenylalanine in Petri dishes. The plates may be transferred to a growth chamber for germination, and when emergence has occurred, the plates may be transferred to the light. Once plantlets have expanded to the first true leaf, they may be transferred to soil and allowed to grow to 3-5 cm in height prior to harvesting of leaves of the upper three nodes and testing for rosmarinic acid content. A typical wild type level of rosmarinic acid in spearmint is about 0.5% (DW). Plants and plant tissues displaying enhanced RA content of between about 1% and 15%, or between about 3% to 9.5% (DW) may be selected, based on their rosmarinic acid content and overall agronomic properties (e.g. growth rate, total biomass), and vegetatively propagated. Bulk quantities of spearmint leaves may be grown for testing of RA content. In one embodiment, the plant material, for instance for testing for RA content, is dried (i.e. its moisture content is reduced from that found in living plants). For instance, after drying at about 35° C. for 96 hours, the moisture content of the plant material is typically about 11.75%.

Alternatively, selection for enhanced rosmarinic acid content in spearmint may be made using spearmint seeds mutagenized with ethyl methane sulfonate or another similar mutagen known in the art. For instance, spearmint seeds may be surface sterilized in a bleach solution, and then washed with sterile water. Seeds may then be transferred to a solution of ethyl methane sulfonate (e.g. 1% (v/v)) and incubated, e.g. for about 18 hours, in the absence of light on a rotary shaker. Seeds may then be washed with sterile water and transferred to sterile Petri planes containing media for further germination and screening.

According to another aspect of the invention, a method of producing a spearmint plant that exhibits enhanced rosmarinic acid content is provided that comprises the steps of: (a) sexually crossing a first parental spearmint plant comprising an enhanced level of rosmarinic acid of more than 77.5 mg/g, 80 mg/g, 85 mg/g, 90 mg/g, or 92 mg/g (DW), up to about 118 mg/g or 150 mg/g, and a second parental spearmint plant that lacks the genetic complement that allows for expression of enhanced levels of rosmarinic acid, thereby producing a plurality of progeny plants; and (b) selecting a progeny plant that comprises enhanced rosmarinic acid content. Breeding methods may additionally comprise the steps of crossing the parental plant comprising enhanced rosmarinic acid to a second parental spearmint plant, and selecting for progeny (F₁ or other generation hybrid) comprising enhanced rosmarinic acid content, for instance by molecular marker DNA genetically linked to the enhanced rosmarinic acid phenotype, and/or the ability to produce more than 77.5 mg/g, 80 mg/g, 85 mg/g, 90 mg/g, or 92 mg/g, up to about 118 mg/g or 150 mg/g rosmarinic acid (DW) in the presence of L-α-bromophenylalanine (e.g. about 0.7 mM) or rosmarinic acid (e.g. about 0.4-1 mM). A selected plant may be propagated vegetatively, for instance by rooted cuttings or by tissue culture (micropropagation).

### I. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Selection of Spearmint Lines with Enhanced Rosmarinic Acid

The screening protocol was performed by screening spearmint seeds (Mentha spicata; Lot 157, Stokes Seeds Ltd., St. Catharines, ON, Canada; country of origin: The Netherlands) in a solution of phenylalanine analogue, L-α-bromophenylalanine, a putative inhibitor of phenylalanine ammonia lyase, the first enzyme in the phenylpropanoid pathway. Alternatively, seeds may be germinated and plants grown in the presence of rosmarinic acid itself (e.g. about 0.4-1 mM) to identify plants that display enhanced levels of rosmarinic acid.

Spearmint seeds (5 g, or about 50,000 seeds) were surface sterilized for 10 minutes in a solution of 100% bleach, then washed three times with sterile water. The seeds were then placed on pre-wetted sterile filter paper soaked in a solution of 0.7 mM L-α-bromophenylalanine in Petri dishes. Plates were transferred to a 30° C. chamber for germination, and when emergence occurred, plates were transferred to the light. Once plantlets expanded to the first true leaf, they were transferred to soil and allowed to grow to 3-5 cm in height prior to harvesting of leaves of the upper three nodes and testing for rosmarinic acid content. A typical wild type level of rosmarinic acid was about 0.5% (DW). Eight plants displaying enhanced RA content of between about 3% to 9.5% (DW) were identified and selected based on their rosmarinic acid content and overall agronomic properties (e.g. growth rate, total biomass), and vegetatively propagated as lines. Among these lines, spearmint line 700B was selected for further use based on its RA content (about 8.8-9.7%) and agronomic properties. Bulk quantities of spearmint leaves were grown at the University of Guelph Arkell Research Station (Arkell, ON, Canada), and passed the Health Canada/Natural Health Product Directorate's purity standards for chemical and microbiological contaminants, (as tested by Nutrasource Diagnostics, Inc, Guelph, ON, Canada).

Screening of spearmint plants grown in the presence of RA (0.4-1 mM) was also performed. After growth and testing of plant material, the nine plants identified by this method with the highest RA content displayed between about 4.5% and 15% RA (i.e. 45 mg/g to 150 mg/g), on a dry weight basis.

Alternatively, selection for enhanced rosmarinic acid content may be made using spearmint seeds mutagenized with ethyl methane sulfonate or a similar mutagen as is known in the art. For instance, spearmint seeds (5 g) were surface sterilized for 10 minutes in a solution of 100% bleach, and then washed three times with sterile water. Seeds were transferred to a sterile 50 mL tube of a 1% (v/v) solution of ethyl methane sulfonate and incubated for 18 hours in the absence of light on a rotary shaker. Seeds were then washed three times with sterile water and transferred to sterile Petri planes containing a water-agar media for further germination and screening. Following testing of rosmarinic acid content, 90 plants selected for further testing displayed a rosmarinic acid content of about 1% to about 10%, DW, compared to the typical level of 0.5%, DW.

Thus the several comparable screens described above successfully identified numerous spearmint plants comprising RA content enhanced above levels previously reported, allowing for development of numerous spearmint lines with enhanced rosmarinic acid content. Without being bound by a particular theory, such screens may operate by assisting in identification of spearmint plants comprising altered phenylpropanoid metabolism, leading to enhanced RA content. Such altered metabolism may be due, for instance, to over expression of a phenylpropanoid pathway synthetic enzyme, the presence of a feedback insensitive enzyme, or reduced metabolism or degradation of rosmarinic acid, etc.

### Example 2

### HPLC Testing of Spearmint Ethanol/Water Extracts for Rosmarinic Add Content

Rosmarinic acid was identified and quantitated using a RP-HPLC method. Briefly, between 5 to 10 mg of ground leaf material (particle size 500 um or less) from indoor grown plant clones was placed in a test tube with 3 mL of 50% ethanol solution and microwaved for 120 seconds (2×60 seconds, 1400 watts). The solution superheats during microwaving. The extract was allowed to cool and then filtered into an HPLC vial (500 μL).

The concentrated extracts were loaded onto a Gilson 234 autosampler attached to a Gilson computer automated HPLC system equipped with two 306 SC-type pumps, dynamic mixer and a 118 dual wavelength UV/VIS detector (Gilson, Inc. Middleton, Wis.) run by the Unipoint 2.1 version software. A Supelco Discovery C18 RP column (250 mm×4.6 mm, 5 mm) (Supelco, Bellefonte, Pa.) was used as the solid phase and a gradient of acetonitrile and 0.1% phosphoric acid was used for separation.

Initial conditions for separation were 25% acetonitrile at 1.3 mL/minute, and a gradient to 32% acetonitrile at 8 minutes, increasing to 95% acetonitrile at 8.1 minutes and maintained for 4 minutes. At 12.1 minutes, acetonitrile was dropped to 25% and equilibrated for another 5 minutes (total time˜17.5 minutes). Retention time for rosmarinic acid was about 6.3 minutes. Rosmarinic acid content was determined by comparison of peak areas to the peak areas resulting from application of an RA standard solution (e.g., Sigma-Aldrich, St. Louis, Mo., product No. 536954) under identical column conditions. Spearmint line 700B was selected based on its rosmarinic acid content and agronomic traits (e.g., growth rate, production of biomass).

### Table 1. RA Content of Individual Potted Plants from Spearmint Clone 700B

TABLE 1
RA content of individual potted plants from spearmint clone 700B
700B Indoor grown material, 5 clones; 50% ethanol extraction

| | | mg/RA per | % RA | % RA Biomass | |
|---|---|---|---|---|---|
| Plant # | Mass | gram DW | Biomass | Mean | stdev |
| Test 1 | | | | | |
| 1 | 0.0048 | 90.03 | 9.03 | 9.41 | 0.66 |
| 2 | 0.0047 | 93.23 | 9.32 | | |
| 3 | 0.0058 | 88.57 | 8.86 | | |
| 4 | 0.0054 | 105.48 | 10.54 | | |
| 5 | 0.005 | 93.23 | 9.32 | | |
| Test 2 | | | | | |
| 1 | 0.0056 | 118.38 | 11.83 | 8.77 | 2.18 |
| 2 | 0.0051 | 91.68 | 9.17 | | |
| 3 | 0.0046 | 93.38 | 9.34 | | |
| 4 | 0.0049 | 74.91 | 7.49 | | |
| 5 | 0.0054 | 60.28 | 6.03 | | |
| Test 3 | | | | | |
| 1 | 0.0055 | 111.36 | 11.14 | 9.69 | 1.05 |
| 2 | 0.0055 | 90.1 | 9.01 | | |
| 3 | 0.0052 | 88.59 | 8.86 | | |
| 4 | 0.0049 | 89.61 | 8.96 | | |
| 5 | 0.0048 | 104.86 | 10.49 | | |

Harvested material from Spearmint 700B clones displayed an average RA content of about 9.3% on a dry weight (DW) basis, or about 92.9 mg RA/g DW plant material, as tested by extraction of RA in 50% ethanol/50% water, by volume. This value is significantly higher than any previously reported RA content for spearmint.

### Example 3

### Preparation of Rosmarinic Acid Water Extract

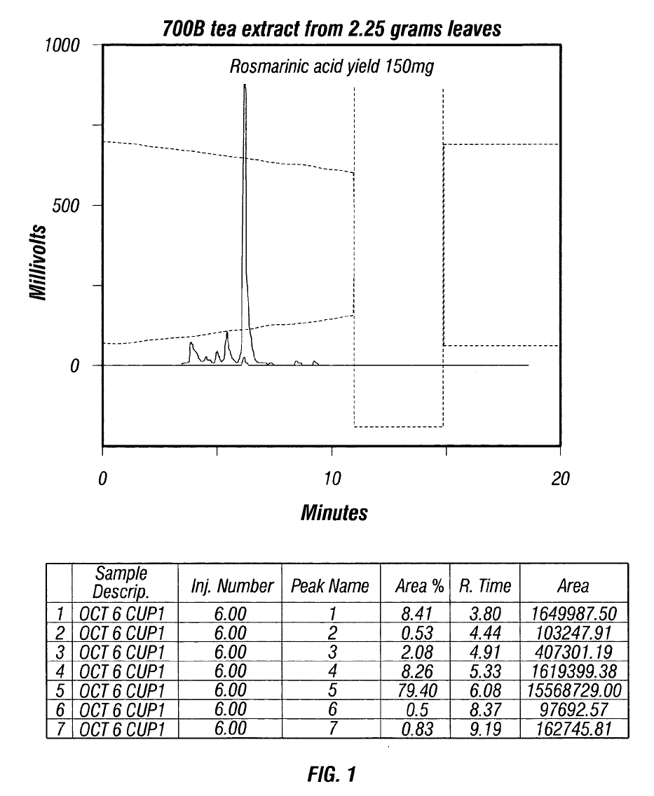

A water extract of Spearmint Line 700B leaves, formulated in a teabag containing 2.25 g DW of crushed leaves, was prepared by steeping a teabag in 250 ml boiling water for 5-10 minutes with occasional stirring. Approximately 150 mg of RA was obtained, as determined by an HPLC method essentially as described in Example 2 except for the initial extraction procedure, or about 67 mg/g DW. The HPLC tracing is shown in FIG. 1. Extraction of RA with water is less efficient than with 50% ethanol/water (v/v), which explains the lower value of RA obtained by this method as compared to that shown in Example 2. Thus knowledge of water extraction values for RA is important for determining the amount of rosmarinic acid that can be expected from a tea (i.e. water extract) preparation.

### Example 4

### Comparison of Rosmarinic Acid Levels Among Spearmint Clones Based On Measurement Methods

Enhanced values of rosmarinic acid content in spearmint were reported previously (McAuley, 2002; Fletcher et al., 2005b). However, these were based upon a spectrophotometric method wherein the absorbance at a wavelength of 333 nm was used. This method does not take into account that other compounds are present, such as other phenolics and flavonoid glycosides, that absorb at this same wavelength. A more accurate way of measuring actual rosmarinic acid content is to use a HPLC method which determines the percentage of rosmarinic acid in the profile that absorbs at 330 nm while separating all the components out individually. In most cases, the rosmarinic acid content in the profiles is between 55-70% of that found by the spectrophotometric method, with the remainder being other compounds. The HPLC method determines the content of rosmarinic acid using standard solutions and by comparing peak areas of those standards to the peak areas of the mint extracts.

Samples from the previous thesis research were kept in dry storage. These were reground and reanalyzed by HPLC (e.g. as per Example 2) following extraction with 50% ethanol/water (v/v). Table 2 below illustrates the rosmarinic acid content of these field samples. The highest concentration of rosmarinic acid, as determined by the present HPLC method (e.g. Example 2) from this series of mint clones was 77.5 mg/g DW which is significantly below the average value for line 700B of about 90 mg/g DW.

### Table 2. RA Contact of Selected Spearmint Clones as Compared by HPLC and Spectrophotometric Methods

TABLE 2
RA content of selected spearmint clones as compared
by HPLC and spectrophotometric methods

| | RA content mg/g DW | McAuley RA values mg/g DW |
|---|---|---|
| Line | HPLC | UV spectroscopy |
| MSH-3 | 53.9* | 59.0 |
| MSH-5 | 59.4 | 122.2 |
| MSH-12 | 48.3 | 58.0 |
| MSH-13 | 55.3 | 96.3 |
| MSH-22 | 71.8 | 85.7 |
| MSH-24 | 42.4 | 82.9 |
| MSH-27 | 71.9 | 77.9 |
| HMS-9 | 59.2 | 73.8 |
| HMS-19 | 71.0 | 81.9 |
| HMS-20 | 39.6 | 107.0 |
| HMS-21 | 77.5 | 110.9 |
| HMS-26 | 42.1 | 103.1 |

### Example 5

### Comparison of Rosmarinic Acid Levels in Teas Made From Commercially Available Spearmint and from Line 700B

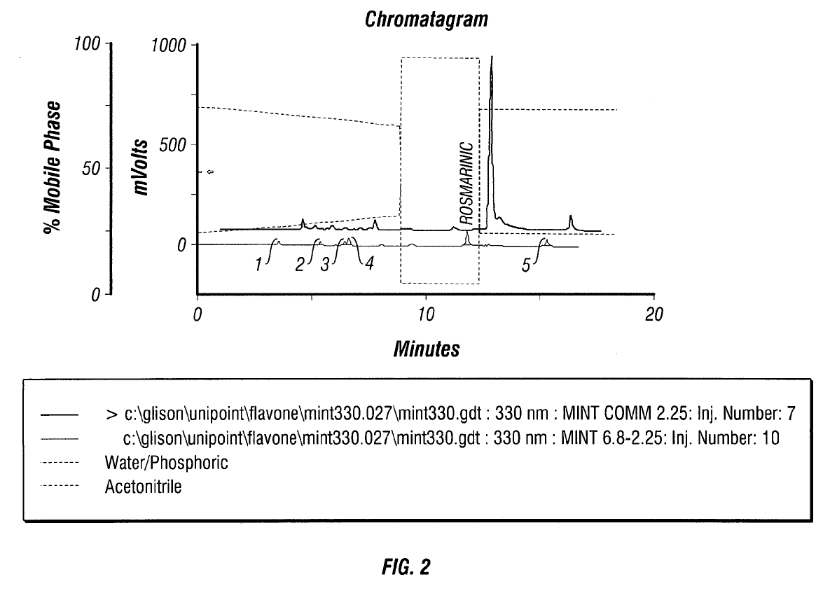

Teas were prepared using teabags comprising about 2.5 g of commercially available spearmint (DistinctlyTea, Waterloo, ON, Canada), or Line 700B spearmint. Teabags were steeped in 250 ml boiling water as described in Example 3. For the Line 700B material, the teabags contained either 2.25 or 2.5 g DW of crushed leaf material. Following HPLC quantitation, essentially as described for instance in Example 3, using a Prevail C18 RP column (Alltech Associates, Deerfield, Ill.) on a Gilson HPLC running Unipoint 2.1 software, the yield of RA in tea from each of these sized teabags was 134 mg and 154 mg per 250 ml cup, respectively. In comparison, the commercially available spearmint teabag, 2.5 g, yielded a tea that comprised only 11 mg RA per 250 ml cup. An HPLC tracing of relative peak sizes is shown in FIG. 2.

### Example 6

### Stability of Rosmarinic Acid in Spearmint 700B Tissues

The level of rosmarinic acid in dried and stored samples of spearmint line 700B tissues was followed over time to determine its stability. Spearmint plant tissue was harvested, dried, and stored, and levels of rosmarinic acid were determined essentially as described by Fletcher et al., (2005a). Plant material was harvested manually from the field and placed in large net bags. The bagged mint sprigs were spread evenly over a grate and dried in a drying oven at a temperature of 35° C. for 96 hours. The stems and other debris were separated from the leaves, and the leaves were crushed to a size suitable for tea. The crushed leaf material was transferred to zip-lock bags and stored at room temperature in the absence of light.

Samples from the growth room and from the field were stored for over 12 months under the described storage conditions and the RA levels were found to remain stable. Thus, for instance, the RA level in a sample of spearmint line 700B plant material was measured shortly after harvest, following drying, and was found to be 80.06 mg/g DW. Following 13 months storage, the RA level was tested again and found to be 78.68 mg/g DW (standard deviation +4.3 mg/g DW; the change is not significant).

Additionally, to study the effect of drying temperature on stability of RA, a sample of spearmint plant material was dried to 11.7% moisture as above for determination of RA content. After 96 hours, a sample of the material was placed into a drying oven and heated to 80° C. for an additional 24 hours, and RA levels were also determined. The amount of RA in the tissues, before and after the 80° C. drying step, remained the same.

### Example 7

### Morphometrics of Mentha spicata Line 700B

Plants of Mentha spicata line 700B were selected based on their elevated rosmarinic acid content and agronomic properties. Table 3 lists morphological traits of Mentha spicata line 700B, and rosmarinic acid content. This line may be propagated vegetatively, for instance by rooting suckers or stem cuttings. The genotype 700B has remained stable and uniform for its morphological characters and showed consistency in performance for various quality attributes, such as rosmarinic acid content, during its evaluation and vegetative multiplication (e.g. see Table 3). Plants of line 700B have not been observed under all possible environmental conditions. Thus, the phenotype may vary somewhat with variations in environment such as temperature and light intensity without, however, any variance in genotype. These characteristics in combination distinguish spearmint line 700B as a new and distinct cultivar.

### Table 3. Traits of Mentha Spicata Line 700B

TABLE 3
Traits of Mentha spicata line 700B

| Trait | Mean/description | Methods |
|---|---|---|
| Leaf arrangement | opposite | family trait |
| Blade length | 4.0 cm (SD 0.46) | (n = 80; range 3.0-4.9 cm) |
| Blade width | 1.9 cm (SD 0.21) | (n = 80; range 1.4-2.4 cm) |
| Petiole length | 1.3 mm (SD 0.05) | (n = 80; range 1.0-3.0 mm) |
| Leaf shape | ovate-lanceolate | visual on largest leaves |
| | leaf tip acute | |
| Leaf margin | serrate (0.5 teeth dist.) | (n = 80; midleaf dentation) |
| Trichomes | very few | visual on largest leaves |
| Underside | few hairs only on main vein | |
| Other traits | upper leaf deeply veined; | |
| | leaves very fragrant | |
| Creeping stem length | none | |
| Erect stem height | 27.6 cm (SD 3.3) | (n = 40; range 22.5-35.5 cm) |
| Internode length | 3.1 cm (SD 0.7) | (n = 80; range 1.2-5.3 cm) |
| Flower colour | pale lilac | visual |
| Flowers per node | 48 (SD 8.9) | (n = 16) |
| Inflorescence type | spike of verticils | (n = 16) |
| Flower length | 3.9 (SD 0.3) | (n = 16) |
| Calyx length | 2.0 (SD 0.3) | (n = 16) |
| Pedicel length | 1.0 (SD 0.1) | (n = 16) |
| Fruit type | 4 nutlets | family trait |
| Fruit colour | dark brown | family trait |
| Rosmarinic acid content | | 92.9 mg/g; range 87.7-118 mg/g |
| (dry weight) | | |

### Example 8

### Effect of Rosmarinic Acid on Allergen-Induced Airway Responses in Mild Allergic Asthmatic Subjects

Subjects. Two subjects (45 and 57 year old, male) were recruited for the study. Inclusion criteria required subjects to be non-smokers with stable, mild atopic asthma, free of other lung disease. Subjects were required to have FEV₁ (forced expiratory volume in 1 second)>70% of predicted, baseline methacholine PC₂₀ (the provocative concentration of methacholine causing a 20% fall in FEV₁)<16 mg/mL, and development of an allergen-induced EAR (at least 20% fall in FEV₁ within 2 h post allergen inhalation) and LAR (at least 15% fall FEV₁ between 3-7 h post allergen inhalation).

Study design. Subjects meeting the inclusion criteria underwent allergen challenge with placebo and rosmarinic acid, in this order, separated by a washout period of at least 4 weeks. Placebo was inhaled PBS on 2 consecutive mornings before challenge. Rosmarinic acid treatment consisted of 7 days of 2 cups of tea per day. Day 6 consisted of pre-dose measurements of airway hyperresponsiveness and sputum cells. On Day 7 allergen inhalation challenge was performed. Measurements of FEV₁ were taken at regular intervals until 7 h after challenge and sputum cells were then collected. On Day 8 subjects underwent 24 h post allergen measurements of airway hyperresponsiveness and sputum cells.

1. Methacholine Inhalation Test

Methacholine inhalation challenge was performed as described by Cockcroft (1985). The test was terminated when a fall in FEV₁ of 20% of the baseline value occurred, and the methacholine PC₂₀ is calculated.

2. Allergen Inhalation Challenge

Allergen challenge was performed as described by O'Byrne (1987), and the concentration of allergen extract (Omega Laboratories, Montreal, QC) for inhalation was determined from a formula described by Cockcroft et al. (1987). The EAR is the maximum % fall in FEV₁ within 2 hours after allergen inhalation, and the LAR is the maximum % fall in FEV₁ between 3 and 7 hours after allergen inhalation.

3. Sputum Analysis

Sputum was induced and processed using the method described by Pizzichini et al. (1996). Differential cell counts were obtained and an aliquot of cells were lysed for quantitative PCR measurements.

4. Results

Methacholine PC20—Airway Hyper-responsiveness. The methacholine PC20 measured before allergen challenges was similar within subjects (Table 4). Both subjects had a reduction in methacholine PC20 measured at 24 h post allergen challenge, and this reduction in methacholine PC20 was similar with placebo and rosmarinic acid treatment (placebo 0.44 mg/ml vs RA 0.38 mg/ml subject #19) (placebo 1.56 mg/ml vs RA 1.27 mg/ml).

### Table 4. Methacholine PC20 and Allergen Dilution

TABLE 4
Methacholine PC20 and allergen dilution

| | | | TIMEPT | | | AG TYPE | |
|---|---|---|---|---|---|---|---|
| ID | NAME | TREATMENT | (HRs) | DATE | PC20 | (SOURCE) | DILUTN |
| 19 | KJK | PLACEBO | 0 | 11 Apr. 2006 | 1.09 | | |
| 19 | KJK | PLACEBO | 7 | 12 Apr. 2006 | | CAT | 128 |
| 19 | KJK | PLACEBO | 24 | 13 Apr. 2006 | 0.44 | | |
| 19 | KJK | TEA | 0 | 11 Jul. 2006 | 1.49 | | |
| 19 | KJK | TEA | 7 | 12 Jul. 2006 | | CAT | 128 |
| 19 | KJK | TEA | 24 | 13 Jul. 2006 | 0.38 | | |
| 20 | LJJ | PLACEBO | 0 | 16 May 2006 | 7.85 | | |
| 20 | LJJ | PLACEBO | 7 | 17 May 2006 | | HDMDP | 64 |
| 20 | LJJ | PLACEBO | 24 | 18 May 2006 | 1.56 | | |
| 20 | LJJ | TEA | 0 | 17 Jul. 2006 | 7.89 | | |
| 20 | LJJ | TEA | 7 | 18 Jul. 2006 | | HDMDP | 128 |
| 20 | LJJ | TEA | 24 | 19 Jul. 2006 | 1.27 | | |

HDMDP—house dust mite, Dermatophagoides pteronyssinus

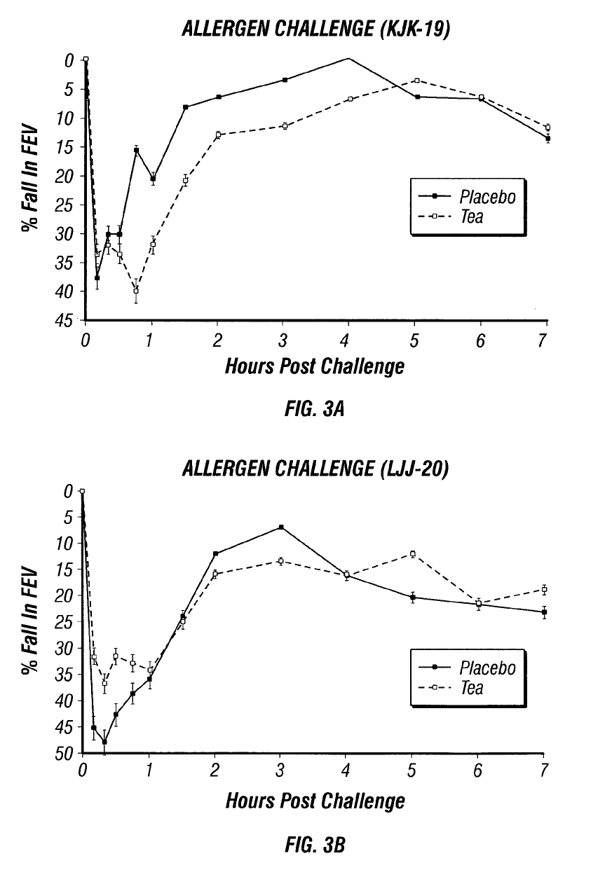

Allergen-Induced Bronchoconstriction. The allergen-induced EAR (Early Asthmatic Response) and LAR (Late Asthmatic Response) was similar between placebo and RA in subject #19. The allergen-induced EAR was lower in subject #20 after RA treatment, however, subject #20 received allergen at 1:128 dilution with RA compared to 1:64 with diluent, as subject could not tolerate a full dose of allergen during RA treatment allergen challenge. Results are shown in FIGS. 3A-3B.

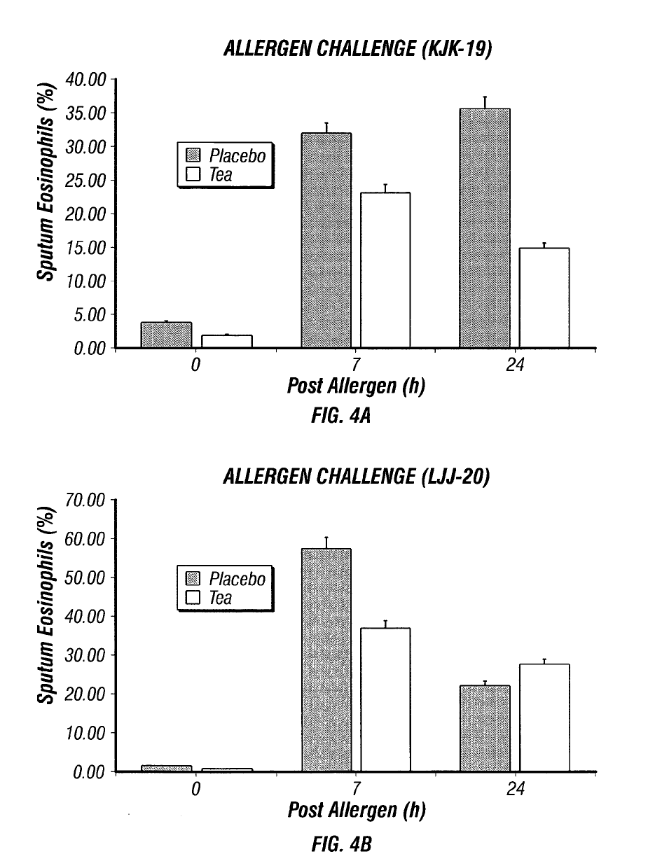

Allergen-Induced Airway Inflammation—Sputum Eosinophils. The % sputum eosinophils appear to be consistently lower post allergen with RA treatment (FIGS. 4A-4B). Rosmarinic acid does not appear to have any effect on the allergen-induced EAR or LAR in this limited sample size. However, the % eosinophils post-allergen was consistently lower with rosmarinic acid treatment. These results suggest that the rosmarinic acid tea would be effective against allergic rhinitis, or other diseases or conditions mediated by the presence of increased number or activity of eosinophils.

### Example 9

### A Double-Blind, Placebo-Controlled, Randomized, Crossover Trial of Mint Tea High in Rosmarinic Acid in Adults with Nasal Polyposis

Background. Nasal polyps are clear, glistening, grape-like structures that occur in two percent of adults and are often associated with Samter's tetrad of asthma, aspirin intolerance and sinusitis. Nasal polyps contain a large number of activated eosinophils—about 20% of the constituents of nasal polyp tissue (Finotto et al., 1994). The impact of treatment on nasal inflammation is a key factor in the evaluation of a new nasal polyp therapy.

The standard treatment for nasal polyp patients is intranasal steroids which, in troublesome cases, may follow a short course of oral steroids. Surgery is reserved for extremely large polyps and those who fail medical treatment. However, many patients tire of using nasal sprays or are troubled by side effects such as nasal irritation or bleeding. So, while all treatments offer some benefit, there is no gold standard. Clinicians and patients often try various therapies and combinations in a ‘hit or miss’ attempt to find relief from symptoms. Any additional treatment options, supported by evidence-based research, would be helpful for both patients and their doctors.

Rosmarinic acid is categorized as a polyphenolic phytochemical, or a ‘plant phenol’ and is found in a variety of plants including the herbs oregano and rosemary, as well as the mints. Peppermint is one of the most widely used single ingredients in herbal teas. It has been found in vitro to have significant antimicrobial and antiviral properties, strong antioxidant and antitumor actions, and some antiallergenic ability (McKay and Blumberg, 2006). Human based research is limited although Takano et. al. (2004) examined the use of rosmarinic acid in seasonal allergic rhinitis. Active treatment significantly decreased the numbers of neutrophils and eosinophils in nasal lavage fluid as well as reducing some subject reported symptoms (Takano et al., 2004).

The research unit has previously studied nasal polyposis. The inventors performed a randomized, placebo-controlled trial of intranasal budesonide for 4 weeks in adults with nasal polyposis. Budesonide treatment improved symptoms, nasal peak inspiratory flow and quality of life as measured by a nasal polyp quality of life questionnaire that the inventors developed. Treatment also resulted in a reduction in blood and nasal lavage eosinophil counts (Keith et al., 1995; Keith et al., 1996). In addition, the inventors performed a randomized, double-blind, placebo-controlled, cross-over study to compare 4 weeks of montelukast 10 mg once daily to placebo. Subjects on active treatment experienced a significant improvement in quality of life and peak nasal inspiratory flow rates (Keith et al., 2003).

A mint tea high in rosmarinic acid has recently been produced. Anecdotal evidence suggests that it may be beneficial for allergic rhinitis if taken prior to allergen exposure (personal communication). A small crossover trial in patients with allergic asthma found a blunting of the sputum eosinophils following allergen challenge (personal communication). This trial aims to study the effects of this mint tea high in rosmarinic acid in adults with bilateral nasal polyps, a condition characterized by chronic eosinophilic inflammation. The control treatment will be a mint tea low in rosmarinic acid.

Methods. Randomized, double-blind, placebo-controlled crossover trial of mint tea high in rosmarinic acid BID vs mint tea low in rosmarinic acid BID in adults with bilateral nasal polyps. Treatment period 1 will be of 4 weeks duration, following a 2 week baseline period. Treatment 1 will be followed by a 4 week washout period and crossover to a further 4 week treatment phase. The nasal polyposis questionnaire, nasal lavage and blood counts will be done at the end of each baseline and treatment period. Nasal peak flow and diary symptoms will be measured daily throughout.

The primary endpoint of this study is to compare the effect of mint tea high in rosmarinic acid with mint tea low in rosmarinic acid by the following measurements:

1. Nasal Polyposis Quality of Life Questionnaire.

2. Nasal patency as assessed by the use of the Clement-Clarke peak nasal inspiratory flow meter (PNIF).

The secondary endpoint of this study is to investigate the effect of mint tea high in rosmarinic acid with mint tea low in rosmarinic acid by the following measurements:

1. Nasal Lavage eosinophils.

2. Peripheral blood eosinophils.

3. Diary symptom score.

4. Nasal polyp size on visual inspection.

Inclusion Criteria—subjects who are male or female aged 18 years or older, who have signed an informed consent agreement, and a history of nasal polyp symptoms during the previous 12 months.

Exclusion Criteria—subjects with severe nasal polyps requiring immediate surgery, presenting with unilateral polyps, have undergone surgery to treat their nasal polyps (nasal polypectomy) within one year prior to visit one, have a known fungal infection of the nose and/or paranasal sinuses, nasal candidiasis, acute or chronic infectious sinusitis of viral or bacterial nature, have had an upper respiratory tract infection within two weeks prior to Visit one or any time between Visit 1 and Visit 2, having cystic fibrosis, Young's syndrome, primary ciliary dyskinesia, known HIV infection or alcohol abuse. clinically significant, uncontrolled evidence of cardiovascular, neurological, hepatic, renal, respiratory, or any other medical condition that may interfere with the study, a recent history (within six months) of a clinically significant psychiatric disorder other than mild depression, have any clinically relevant deviation from normal in the general physical examination, have received any depot, systemic or oral corticosteroid in the previous three months prior to the start of the study, unable to cease treatment with intranasal steroids four weeks prior to Visit one, known hypersensitivity to mint, females who are pregnant or lactating or are likely to become pregnant during the study or are less than 8 weeks postpartum. Women of childbearing age may be included if in the opinion of the investigator, they are taking adequate contraceptive measures, unable to follow the instructions within this protocol or known inability to attend all clinic visits within the intervals stated, have participated in a clinical trial involving an investigational or marketed drug within four weeks of visit one, and those who are allergy skin test positive to a seasonal allergen which will be present when performing the trial, that has caused, within the past 2 years, a clinically significant deterioration in nasal symptoms.

Disallowed Medications—intranasal steroids within 4 weeks of Visit 1, depot, systemic, or oral corticosteroids within 3 months of Visit 1 (inhaled and topical corticosteroids are allowable), oral leukotriene receptor antagonists within 2 weeks of Visit 1, antibiotics used to treat an acute infection within 1 month of Visit 1, NSAIDS (such as Celebrex®, Ponstan®), ibuprofen and ASA within 24 hours of Visit 1, nasal or oral decongestants such as Otrivin® or Sudafed® within 24 hours of Visit 1, nasal, ophthalmic, or inhaled cromolyn, or nedocromil within 2 weeks of Visit 1, nasal saline sprays (such as Hydra Sense®) within 24 hours of Visit 1 (saline nasal spray will be dispensed at Visit 1 as study rescue medication).

Allowed Medications—inhaled corticosteroids for treatment of asthma, antihistamines: short acting and/or long acting ocular or oral antihistamines may be used on an “as needed” basis only, immunotherapy (subject must be on a stable maintenance dose during the 6 months prior to Visit 1 and throughout the course of the study), antibiotics (such as Minocin®) if on a maintenance dose that will continue throughout the study, acetaminophen and codeine in monosubstance formulations, medications used to treat concurrent disorders that do not affect nasal symptoms, at constant dosage regimens, will be allowed unless specifically excluded. The treatment must have been initiated at least one month prior to Visit 1.

Study Design. The planned duration of enrollment (from first patient screened to last patient randomized) is 8 months. The planned duration of the entire study (baseline, treatment, washout, treatment) is approximately 12 months. Subject will start taking the study treatment by drinking one cup of study tea every evening for approximately four weeks. After evaluation, and a four-week treatment-free period, the subject will again start taking the study treatment by drinking one cup of study tea every morning and one cup of tea every evening for approximately four weeks. Another four-week treatment-free period completes the trial.

The study tea will be provided in 2.5 mg tea bags. Two cups of active tea will provide 300 mg of rosmarinic acid per day. Two cups of placebo tea will provide 20 mg of rosmarinic acid per day. The study tea will be brewed with 250 ml of boiling water and allowed to steep for 10 minutes. The tea may be sweetened with sugar or honey and subjects must report on the diary card whether sweetener was used and in what amount.

There are 2 double-blind cross-over treatment periods. At Visit 2, subjects who have met all the inclusion entrance criteria will be assigned a treatment number and randomized to Treatment A (either mint tea high in rosmarinic acid twice a day or mint tea low in rosmarinic acid twice a day). At the end of 4 weeks, the subjects will have a 4 week washout period. At Visit 4, subjects will receive their crossover treatment, i.e. Treatment B. Each group will receive study medication in a “double-blind” manner.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 6,140,363

U.S. Patent Publication 2006/0134236

Alkam et al., Behay. Brain Res., 180(2):139-145, 2007.

Baba et al., Eur. J. Nutr., 43:1-9, 2004b.

Baba et al., Life Sciences, 75:165-178, 2004a.

Cockcroft et al., Am. Rev. Respir. Dis., 135:264-267, 1987.

Cockcroft, In: Airway responsiveness: measurement and interpretation, Hargreave and Woolcock (Eds.), Mississauga: Astra Pharmaceuticals Canada Ltd, 22-28, 1985.

Finotto et al., Clin. Exp. Immunol., 95:343-350, 1994.

Fletcher et al., ISHS Acta Horticulturae 680:31-40, 2005b.

Fletcher et al., J. Sci. Food Agric., 85:2429-2436, 2005a.

Hug, “Meiji Seika Kaisha Confirms Perilla—derived Rosmarinic Acid's Effectiveness Against Hay Fever”, http://wwwjapancorp.net/Article.Asp?Art_ID=9499, 2005.

Inoue et al., Int. J. Molec. Med., 16:315-9, 2005.

Iuvone et al., J. Pharm. Exp. Ther., 317:1143-1149, 2006.

Keith et al., Allergy Clin. Immunol. Int: J. World Allergy Org., Supp 1:185, 2003.

Keith et al., J. Allergy Clin. Immunol., 95:204, 1995.

Keith et al., J. Allergy Clin. Immunol., 97:192, 1996.

Konishi et al., J. Agr. Food Chem., 53:4740-4746, 2005.

Kosar et al., J. Agric. Food Chem., 52:5004-5010, 2004.

McAuley, In: C. Phenolic compounds in Mentha spicata: quantification identification and antioxidant activity, Ph.D. thesis, University of Guelph, 2002.

McKay and Blumberg, Phytother. Res., 619-633, 2006.

Nakazawa and Ohsawa, J. Nat. Prod., 61:993-996, 1998.

O'Byrne et al., Am. Rev. Respir. Dis., 136:740-751, 1987.

Osakabe et al. Biofactors, 21:127-31, 2004.

Petersen and Simmonds, Phytochemistry, 62:121-127, 2003.

Pizzichini et al., Am. J. Respir. Crit. Care Med., 154:308-317, 1996.

Renzulli et al., J. Appl. Toxicol., 24:289-296, 2004.

Ritschel et al., Methods Find Exp. Clin. Pharmacol., 11:345-352, 1989.

Sanbongi et al., Clin. Exp. Allergy, 34(6):971-977, 2004.

Sanbongi et al., Free Rad. Biol. Med., 34:1060-1070, 2003.

Shasany et al., J. Hered. 86:542-549, 2005.

Takano et al., Exp Biol. Med., 229:247-254, 2004.

Takano et al., Exp. Biol. Med., 229:247-255, 2004.

Tewtrakul et al., Phytother. Res., 17:232-239, 2003.

Wang et al., Food Chem., 87:307-311, 2004.

Yun et al., Transplantation, 75:1758-1760, 2003.

1. A plant tissue of a spearmint (Mentha spicata) plant, wherein the tissue comprises more than 77.5 mg/g rosmarinic acid to about 150 mg/g rosmarinic acid, on a dry weight basis.

2. The tissue of claim 1, wherein the tissue comprises a leaf, a stem, a flower, a seed, or a root, or parts or combinations thereof

3. The tissue of claim 1, comprising from about 87 mg/g to about 150 mg/g rosmarinic acid on a dry weight basis.

4. The tissue of claim 1, defined as obtained from a plant containing genetic means for the expression of said more than 77.5 mg/g rosmarinic acid found in spearmint line 700B.

5. A spearmint plant comprising the tissue of claim 1.

6. A part of the plant of claim 5.

7. The part of claim 6, further defined as a leaf, stem, pollen, flower, seed, root, or cell.

8. The tissue of claim 1, wherein the tissue is dried and/or crushed or ground.

9. A teabag, tea leaves, caplet, tablet or nutraceutical formulation comprising the plant tissue of claim 8.

10. A tissue culture comprising cells of the plant of claim 5.

11. A tea produced from the tea leaves or teabag of claim 9, which tea comprises at least about 90 mg or more of rosmarinic acid per 250 ml.

12. A method for producing rosmarinic acid, comprising cultivating the plant of claim 5.

13. The method of claim 12, further comprising isolating rosmarinic acid from the plant.

14. A method for producing rosmarinic acid, comprising growing a tissue culture comprising cells of the plant of claim 5.

15. A method of producing a beverage comprising rosmarinic acid, comprising contacting the plant tissue of claim 1 with an edible liquid and allowing rosmarinic acid from the tissue to dissolve in the liquid.

16. The method of claim 15, wherein the liquid comprises alcohol, water, or a combination thereof.

17. A method for providing rosmarinic acid to a subject, comprising administering to the subject the tissue of claim 1 or a composition comprising rosmarinic acid there from.

18. The method of claim 17, wherein the tissue is further defined as obtained from a plant according to claim 5.

19. The method of claim 17, wherein the subject is defined as comprising an inflammatory or infectious disease, and wherein the rosmarinic acid treats the inflammatory or infectious disease.

20. The method of claim 17, wherein the composition comprises a beverage.

21. The method of claim 20, wherein the beverage is a tea.

22. The method of claim 19, wherein the subject suffers from nasal polyps, asthma, allergy, hay fever, seasonal allergic rhinitis, perennial allergic rhinitis, allergic rhinoconjunctivitis, eosinophilia, hypersensitivity, allergic conjunctivitis, eczema, food allergy, dermatitis, Alzheimer's Disease, or wherein the rosmarinic acid acts as an immunosuppressant, hepato- or neuro-protective, antibacterial, or antiviral agent.

23. A method for reducing the number of eosinophils at a site in a subject comprising administering to the subject the tissue of claim 1 or a composition comprising rosmarinic acid there from.

24. A method of producing seed, comprising crossing the plant of claim 5 with itself or a second spearmint plant.

25. A method of producing a spearmint plant comprising: preparing a progeny plant derived from the plant of claim 5 by crossing the plant with a second spearmint plant, and obtaining a progeny plant according to claim 5.

26. The method of claim 25 wherein a plurality of progeny plants are produced.

27. The method of claim 26, defined as comprising selecting one or more progeny plants based on rosmarinic acid content.

28. The method of claim 25 wherein the progeny plant is vegetatively propagated.

29. A method for improving memory or impairing memory loss in a subject comprising administering to the subject the tissue of claim 1 or a composition comprising rosmarinic acid there from.
